(19) United States
(12) Patent Application Publication
(10) Pub. No.: US 2010/0268311 A1
(43) Pub. Date: Oct. 21, 2010

(54) Method for Implanting Electrode on Nerve

(76) Inventors: Ralph Cardinal, White Bear Lake, MN (US); Jason Skubitz, Arden Hills, MN (US); Jason Shiroff, Minneapolis, MN (US); Johann Neisz, Coon Rapids, MN (US)

Correspondence Address: ST. JUDE MEDICAL NEUROMODULATION DIVISION, 6901 PRESTON ROAD, PLANO, TX, 75024, US

(21) Appl. No.: 12/761,956

(22) Filed: Apr. 16, 2010

Related U.S. Application Data
(60) Provisional application No. 61/170,377, filed on Apr. 17, 2009.

(51) Int. Cl. A61N 1/05

(52) U.S. Cl. 607/118

(57) ABSTRACT

Methods and devices for implanting an electrode near a nerve covered by a tissue layer or layers. Methods can include cutting through a tissue layer covering the nerve to form at least two exposed pleural tissue layer edges. The nerve can be freed from any tissue around the nerve and the electrode placed around the nerve. The pleura edges can be drawn toward each other to cover the nerve and the edges secured together. Some methods form two tissue flaps which are pulled away from each other to expose the nerve. Other methods form a single tissue flap which extends over the nerve and is pulled back to expose the nerve. The now covered nerve is protected against movement of tissues near the now covered nerve. Such methods find one use in placing electrodes near the splanchnic nerves in the thoracic cavity, where the lung and diaphragm may contact an exposed electrode.

### CROSS REFERENCE TO RELATED APPLICATION

This application claims priority from U.S. provisional application 61/170,377, filed Apr. 17, 2009, which is herein incorporated by reference in its entirety.

### TECHNICAL FIELD

The present invention is related generally to implantable medical devices. More specifically, the present invention is related to neurostimulation electrodes placed near nerves and under overlying tissue layers.

### BACKGROUND

Electrodes for stimulating and sensing nerves and other tissues are well known. Electrodes have been used in cardiac pacing, sensing, and defibrillation applications. Deep brain stimulation electrodes have also been used in epilepsy and other applications. Spinal electrodes have been used to mask pain. Each application has brought its own set of unexpected problems requiring novel approaches.

Stimulation of sympathetic nervous system nerves in the thoracic cavity has proven more difficult than first expected. The sympathetic chain runs along either side of the spinal column from the neck down past the diaphragm, on the back wall of the thoracic cavity. The sympathetic chain branches off into other nerves. Of particular interest to applicants are the splanchnic nerves, some of which innervate the gut. These nerves are of interest because of their possible use in treating obesity.

Peripheral nerve applications have previously used nerve cuff electrodes, which surround a nerve and are used to stimulate and/or sense the nerve. Use of cuff electrodes on the Greater Splanchnic Nerve (GSN) has required cutting through the overlying pleura and bluntly dissecting the GSN from adhering tissue binding the nerve. The cuff electrode can then be slipped around the nerve and sutured or otherwise secured in place. The cuff is still exposed, which would not present a problem in some applications. In this application however, the lungs may be in contact with the cuff. Lung movement could impart undesirable movement to the cuff. As the GSN is located just posterior to the diaphragm, it also may be contact with the cuff. Pressures may be brought to bear on the cuff and/or lack of movement may allow for adhesions to form on the cuff.

What would be desirable are improved methods for protecting an electrode placed on nerves normally covered by tissue within the thoracic cavity.

### SUMMARY

Methods and devices for implanting an electrode near a nerve covered by a tissue layer or layers. Methods can include cutting through the tissue layers covering the nerve to form at least one free pleural tissue layer flap having an edge. The nerve can be freed from any tissue under and around the nerve and the electrode placed around the nerve. The pleural edges can be drawn toward each other to cover the nerve and electrode and the edges secured together. The now covered nerve is protected against movement of tissues near the now covered nerves. Such methods find one use in placing electrodes near the splanchnic nerves in the thoracic cavity, where lung and diaphragm movement may nudge an exposed electrode.

In some methods, the tissue cutting includes fine dissection and freeing the nerve includes using blunt dissection to free the nerve from tissue around the nerve. Some methods include removing some tissue under the nerve. Some electrodes have a tubular shape portion and the inserting includes enclosing the nerve region in the tubular shape portion. Securing can include suturing the tissue edges together.

Securing may include suturing the tissue edges together such that the tissue free edges are pulled together to at least substantially hide the sutures extending through the tissue free edges. Cutting may include cutting the tissue layer on opposite sides of the nerve, such that a free tissue layer strip remains over the nerve and the two free edges are disposed on either side of the free tissue layer strip.

Methods according to the present invention may be used to particular advantage with the thoracic splanchnic nerves, the Greater Splanchnic Nerve, and nerves in the sympathetic chain. In some methods, the electrode is coupled to a lead body, and at least part of the lead body near the electrode is at least partially covered by the tissue. In some methods the electrode part of the lead body near the electrode includes a portion nearest the electrode which is substantially aligned with the electrode and which further includes an angled portion located further way from the electrode.

### DESCRIPTION OF DRAWINGS

FIG. 1 is a photographic view of a nerve cuff electrode in place around an exposed Greater Splanchnic Nerve (GSN) in the thoracic cavity.

FIG. 2 is a photographic view of the cuff electrode of FIG. 1 in place around the (GSN), having forceps holding one edge of pleura tissue which had previously covered the GSN.

FIG. 3 is a photographic view of the cuff electrode of FIG. 1 in place around the (GSN), having suture material being passed through one edge of the pleura using a needle.

FIG. 4 is a photographic view of the cuff electrode of FIG. 1 in place around the (GSN), having suture material passed through both opposing edges of the pleura.

FIG. 5 is a photographic view of the cuff electrode of FIG. 1 in place around the (GSN), having more suture material passed across over the cuff and through both opposing edges of the pleura.

FIG. 6 is a photographic view of the cuff electrode of FIG. 1 in place around the (GSN), having still more suture material passed across over the cuff and through both opposing edges of the pleura.

FIG. 7 is a photographic view of the cuff electrode of FIG. 1 in place around the (GSN), having still more suture material passed across over the cuff and through both opposing edges of the pleura.

FIG. 8 is a photographic view of the cuff electrode of FIG. 1 in place around the (GSN), having still more suture material passed across over the cuff, with the opposing edges of the pleura partially approximated, or brought together.

FIG. 9 is a photographic view of the cuff electrode of FIG. 1 in place around the (GSN), the opposing edges of the pleura more closely partially approximated.

FIG. 10 is a photographic view of the cuff electrode of FIG. 1 in place around the (GSN), the opposing edges of the pleura more even more closely partially approximate, to cover or almost cover the GSN.

FIG. 11 is a schematic view of a cuff electrode having an angled and/or canted strain relief covered by the approximated pleura tissue.

FIG. 12 is a photographic view of another nerve cuff electrode in place around an exposed Greater Splanchnic Nerve (GSN) in the thoracic cavity, using minimally invasive techniques to form a single pleural flap.

FIG. 13 is a photographic view of the cuff electrode of FIG. 12 in place around the (GSN), showing a second suture attachment point being made.

FIG. 14 is a photographic view of the cuff electrode of FIG. 12 in place around the (GSN), showing the second suture attachment point being finished.

### DETAILED DESCRIPTION

The present invention can be used in minimally invasive procedure as well as other, more invasive procedures. Some embodiments of the present invention can be used to particular advantage in placing an electrode, for example a cuff electrode, around a splanchnic nerve in the thoracic cavity. In one such method, a cuff electrode is placed around the greater splanchnic nerve in the thoracic cavity.

In one procedure, minimally invasive ports are cut into the thoracic cavity, some through the intercostal spaces. The patient is put under anesthesia, a breathing tube inserted, and a lung deflated to provide better access to the posterior wall of the thoracic cavity just superior to the diaphragm. The diaphragm may be retracted as well. The proper minimally invasive tools such as a video scope, light, forceps, and the like can be inserted through the ports to the proper locations in the thoracic cavity.

The photos below show an actual placement of a cuff around a nerve in a canine.

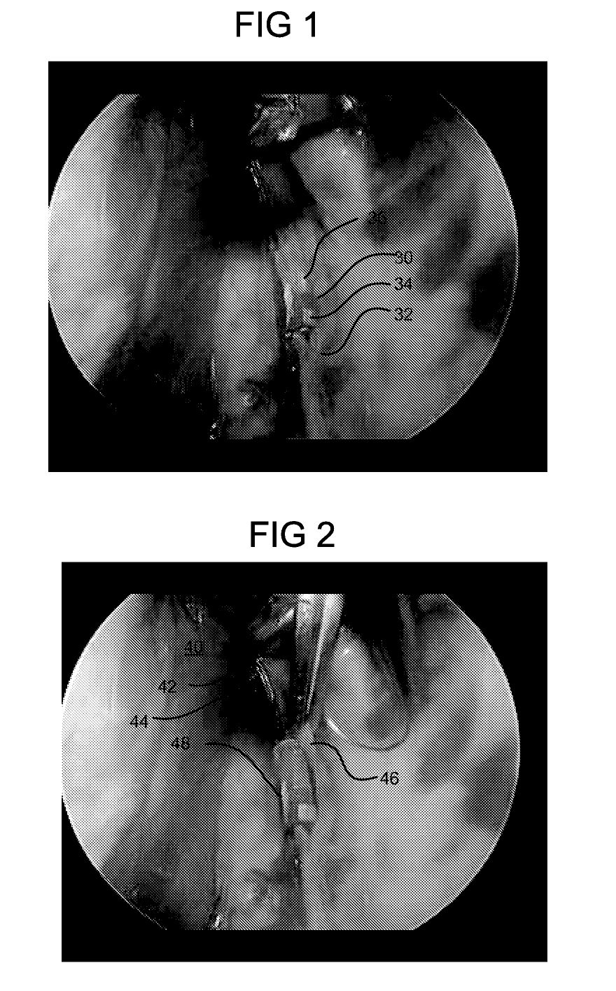

FIG. 1 shows a nerve cuff electrode 30 in place around an exposed Greater Splanchnic Nerve (GSN) 32 in the thoracic cavity. Cuff electrode 30 includes two electrodes 34 and 36. The pleura has already been slit to expose the GSN, the GSN bluntly dissected to tease it from the surrounding tissue, and the cuff put in place around the GSN. In some methods, the dissected area is washed with antibiotic and/or other solutions to leave behind a sterile area.

FIG. 2 shows cuff electrode of FIG. 1 in place around the (GSN) within the thoracic cavity wall 40, having forceps 42 holding one edge flap 46 of pleura tissue which had previously covered the GSN. The opposite edge of the pleura 48 is shown, as is the lead body 44, extending away from the cuff.

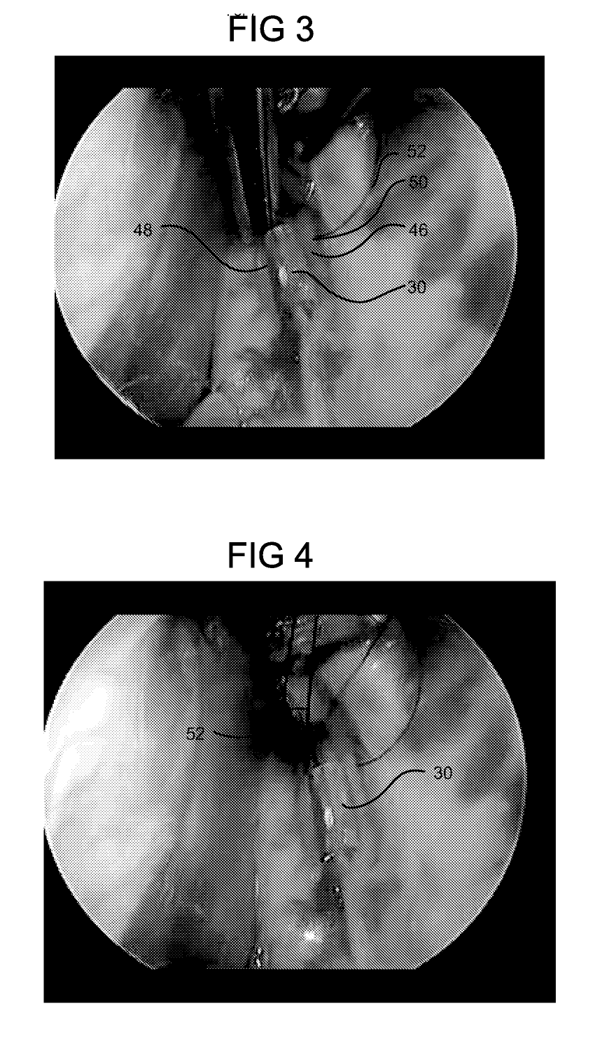

FIG. 3 shows cuff electrode 30 of FIG. 1 in place around the (GSN), having suture material being 52 passed through one edge flap 46 of the pleura at location 50 using a needle.

FIG. 4 is a photographic view of the cuff electrode 30 of FIG. 1 in place around the (GSN), having suture material 52 passed through both opposing edges of the pleura.

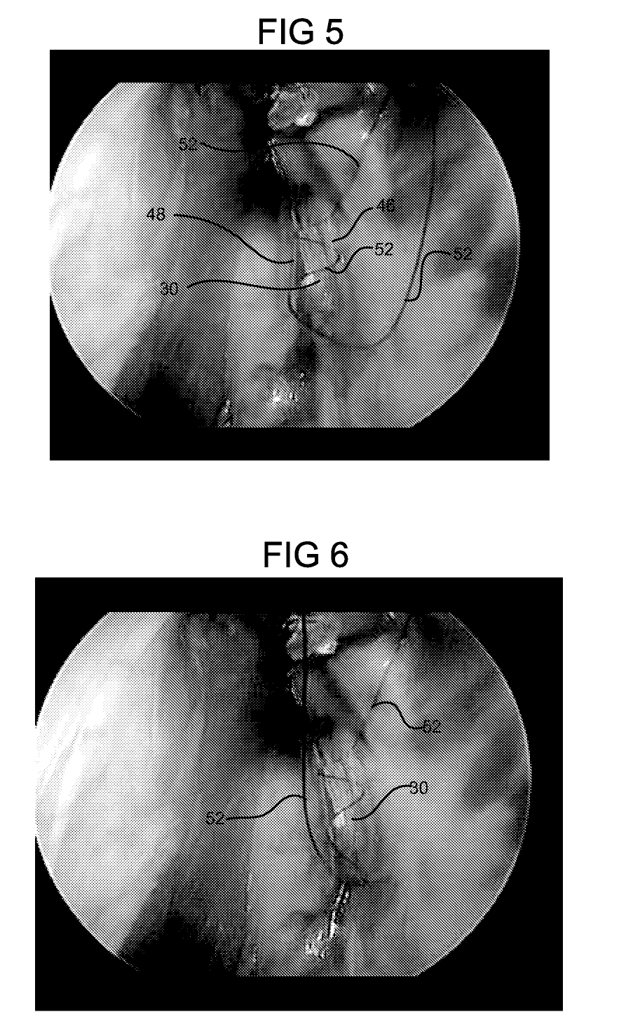

FIG. 5 is a photographic view of the cuff electrode 30 of FIG. 1 in place around the (GSN), having more suture 52 material passed across over the cuff 30 and through both opposing edges 46 and 48 of the pleura.

FIG. 6 is a photographic view of the cuff electrode 30 of FIG. 1 in place around the (GSN), having still more suture material 52 passed across over the cuff and through both opposing edges of the pleura.

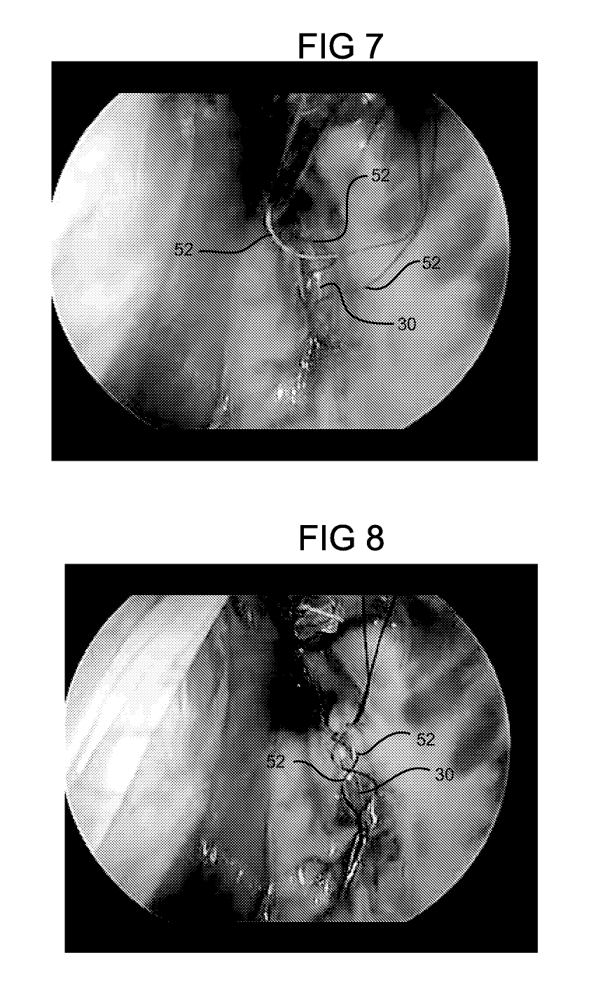

FIG. 7 is a photographic view of the cuff electrode 30 of FIG. 1 in place around the (GSN), having still more suture material 52 passed across over the cuff and through both opposing edges of the pleura.

FIG. 8 is a photographic view of the cuff electrode 30 of FIG. 1 in place around the (GSN), having still more suture material 52 passed across over the cuff, with the opposing edges of the pleura partially approximated, or brought together.

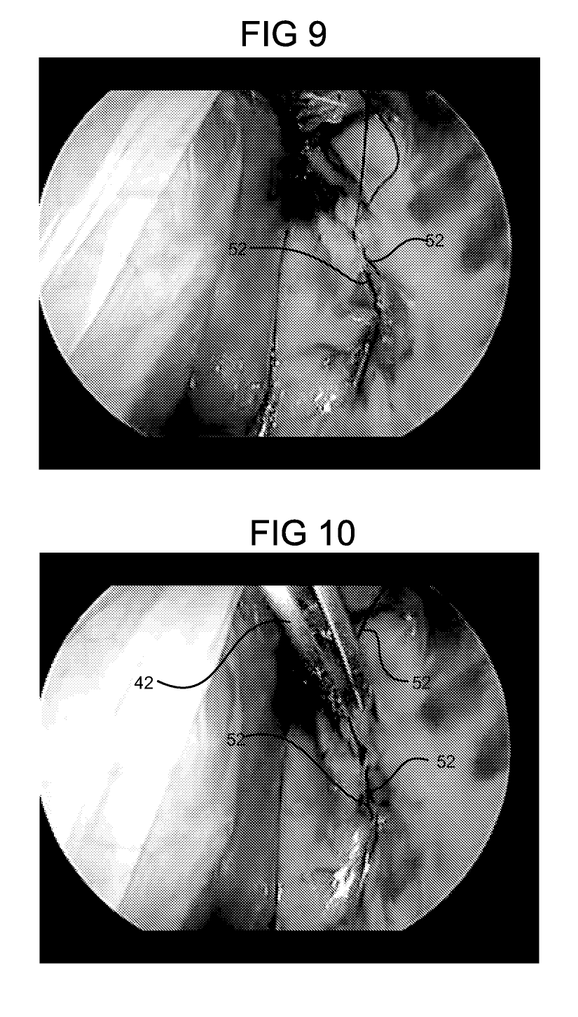

FIG. 9 is a photographic view of the cuff electrode of FIG. 1 in place around the (GSN), the opposing edges of the pleura more closely partially approximated due to the suture material 52 being drawn more tightly.

FIG. 10 is a photographic view of the cuff electrode of FIG. 1 in place around the (GSN), the opposing edges of the pleura more even more closely partially approximate, to cover or almost cover the GSN due to the suture material 52 being drawn more tightly by forceps 42.

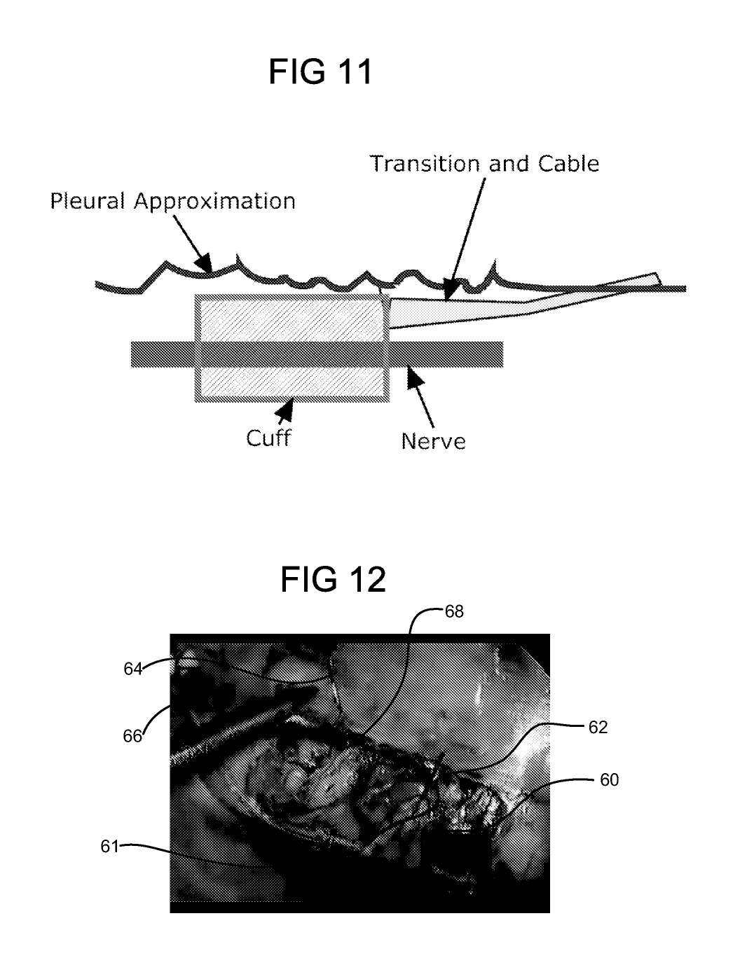

FIG. 11 is a schematic view of a cuff electrode having an angled or canted strain relief covered by the approximated pleura tissue. Cuff electrodes often have the lead body carrying the wire conductors extending linearly from away from the cuff, at least near the cuff. This may not present a problem in other applications. In the present application, it may be desirable to have the lead body remain protected under the tissue immediately near the cuff. This provides additional strain relief and allows the cuff to remain axially aligned with the nerve.

FIG. 12 is a photographic view of another nerve cuff electrode in place around an exposed Greater Splanchnic Nerve (GSN) in the thoracic cavity, using minimally invasive techniques to form a single pleural flap. In this method, a single flap 60 has been formed, already attached to an exposed tissue edge with a first knot 62. The lead body 61 may be seen extending under flap 60. A first minimally invasive forceps 66 and needle 64 are being used to form a second knot using suture material 68. The single flap 60 has been outlined in a dashed line to better show the flap edges against the background of the tissue underneath the flap.

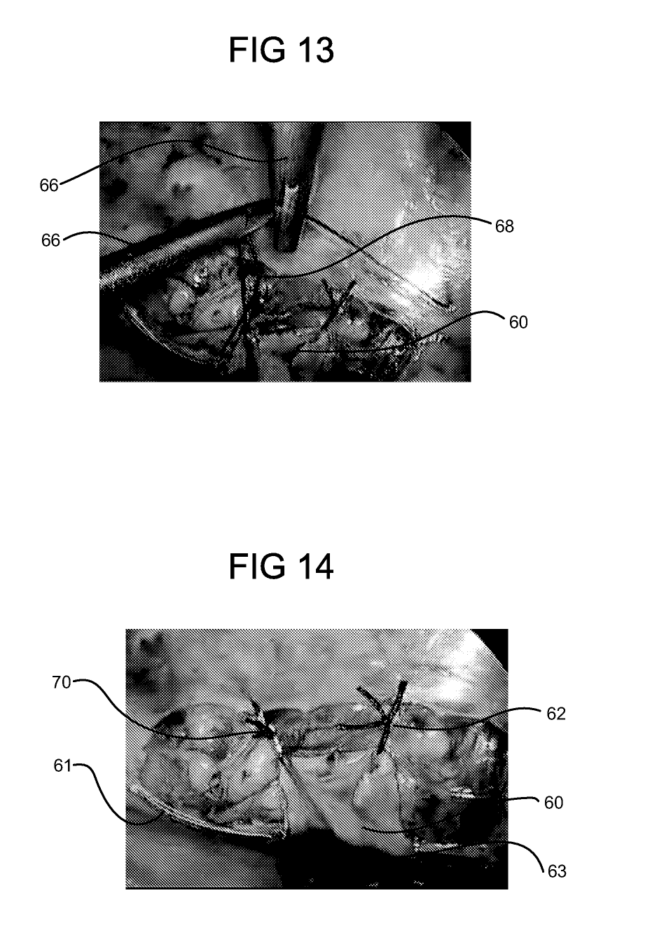

FIG. 13 is a photographic view of the cuff electrode of FIG. 12 in place around the (GSN), showing a second knot being made using suture material 68 and forceps 66. Flap 60 has suture material 68 pulled through the flap.

FIG. 14 is a photographic view of the cuff electrode of FIG. 12 in place around the (GSN), showing the second knot 70 and first knot 62, securing flap 60 over the nerve cuff. The lead body 61 extends under flap 60 and the approximate position of the covered nerve cuff is indicated at 63.

What is claimed is:

1. A method for implanting an electrode near a nerve covered by a tissue layer, the method comprising:
cutting through a tissue layer covering the nerve to form at least two tissue layer exposed edges;
freeing a region of the nerve from tissue surrounding the nerve;
inserting the electrode under the tissue near the nerve;
drawing the tissue exposed edges toward each; and
securing the tissue exposed edges toward each other to cover the nerve.

2. The method as in claim 1, in which the tissue cutting includes fine dissection.

3. The method of claim 1, in which the freeing includes using blunt dissection to free the nerve from tissue around the nerve.

4. The method of claim 1, in which the method also includes removing some tissue under the nerve.

5. The method of claim 1, in which the electrode has a tubular shape portion and in which the inserting includes enclosing the nerve region in the tubular shape portion.

6. The method as in claim 1, in which the securing includes suturing the tissue edges toward each other.

7. The method as in claim 1, in which the securing includes suturing the tissue edges toward each other such that the tissue edges are pulled together to at least substantially hide the sutures extending through the tissue edges.

8. The method of claim 1, in which the cutting includes cutting the tissue layer on opposite sides of the nerve, such that a tissue layer strip remains over the nerve and the two tissue edges are disposed on either side of the tissue layer strip.

9. The method of claim 1, in which the nerve is a thoracic splanchnic nerve.

10. The method of claim 1, in which the nerve is the Greater Splanchnic Nerve.

11. The method of claim 1 in which the nerve is a nerve in the sympathetic chain.

12. The method of claim 1 in which the electrode is coupled to a lead body, and in which at least part of the lead body near the electrode is at least partially covered by the tissue.

13. The method of claim 12 in which the electrode part of the lead body near the electrode includes a portion nearest the electrode which is substantially aligned with the electrode and in which further includes an angled portion located further way from the electrode.

14. The method of claim 1 in which the cutting through the tissue layer to form the tissue exposed edges is performed so as to form at least two tissue flaps.

15. The method of claim 1 in which the cutting through the tissue layer to form the tissue exposed edges is performed so as to form a single tissue flap.
